(19) United States
(12) Patent Application Publication
(10) Pub. No.: US 2004/0258700 A1
(43) Pub. Date: Dec. 23, 2004

(54) Vaccine formulation with a preservative

(76) Inventor: Tine Holland Frimann, Praesto (DK)

Correspondence Address: MUSERLIAN, LUCAS AND MERCANTI, LLP, 475 PARK AVENUE SOUTH, 15TH FLOOR, NEW YORK, NY, 10016, US

(21) Appl. No.: 10/601,719

(22) Filed: Jun. 23, 2003

(51) Int. Cl. A61K 39/12; A61K 47/00; A61K 45/00; A61K 39/38; A61K 39/00

(52) U.S. Cl. 424/184.1; 424/278.1

(57) ABSTRACT

There is a safety concern about a number of vaccine preservatives in use today, such as formaldehyde, phenol, organic mercury (thiomersal) or betapropiolactone for safety reasons. In a few vaccines, 2-phenoxyethanol has been used as a vaccine perservative, but has not gained widespread use, because the preservative effect is inferior to e.g. thiomersal. Applicant has found that the preservative effect of a mixture of 2-phenoxyethanol and para hydroxybenzoates, (e.g. methyl-, ethyl-, propyl- or butyl-, p-hydroxybenzoate) is more effective than 2-phenoxyethanol alone and that this mixture, called TF-1, exerts a preservative effect which is comparable to that of thiomersal or chloroform in vaccines. Although the preservative effect of TF-1 is satisfactory for securing the microbial stability of the vaccine, none of the constituents in TF-1 are considered as critical in terms of toxicity for the vaccinated animals or humans as chloroform or organic mercury compounds, traditionally used for the same purpose.

### FIELD OF THE INVENTION

The present invention relates to a vaccine formulation and in particular a vaccine formulation comprising an immunogen, an adjuvant and a preservative.

### BACKGROUND OF THE INVENTION

Vaccines are administered to birds and mammals, including man, in order to elicit an appropriate immune response to resist or eliminate a certain pathogen. Besides from having a certain level of potency, stability and ability to enhance an immune effect, a vaccine formulation needs to be microbiologically stable, i.e., the vaccine formulation should not support growth of microorganisms and fungi during long time storage of the formulation as well as during use of the vaccine formulation. The use of certain well-known preservatives such as thiomersal, chloroform and formaldehyde has been questioned. Thiomersal, which contains mercury, is claimed to cause autism in children and the use of chloroform in veterinary vaccines has been prohibited due to the risk of too high residual concentrations in the slaughter animals. Thus, there is a need for a vaccine formulation comprising preservatives that are safe as well as effective.

### DESCRIPTION OF RELATED ART

Preservatives suitable for vaccines should be environmentally safe, effective against bacteria as well as yeast and other fungi and devoid of negative impact on the immunogenic effect of the vaccine. Moreover, in the case of veterinary vaccines which are used in animals for consume no hazardous residual amounts of the preservative may be found in the carcass.

Phenoxyethanol is known to have been used in a concentration of 2.5 mg/ml in a vaccine against hepatitis A for human use.

A mixture of methyl and propyl p-hydroxybenzoate is known to have been used in a sedative (Sedaperone© Vet.) for intravenous use in pigs, but has never found use in vaccines.

### DEFINITIONS

The term “paraben esters” refers to methyl, ethyl, propyl or butyl p-hydroxybenzoate, also systematically termed methyl 4-hydroxybenzoate, ethyl 4-hydroxybenzoate, propyl 4-hydroxybenzoate or butyl 4-hydroxybenzoate, respectively.

The term “immunogen” or an antigen refers to a substance that causes the formation of an antibody or elicits a cellular response resulting in immunity of the host towards the pathogen.

The term “adjuvant” refers to a substance in a vaccine that is able to increase the body's immune response to said vaccine.

### DESCRIPTION OF THE DRAWINGS

Tolerance tests on pilot vaccine with TF-1 towards the germ S. aureus. The tests were made after the vaccine was kept in 0, 3, 12, 24 or 39 months at 4-6° C. The data are normalized and based on the results from the test of the vaccine kept in 0 months.

### DESCRIPTION OF THE INVENTION

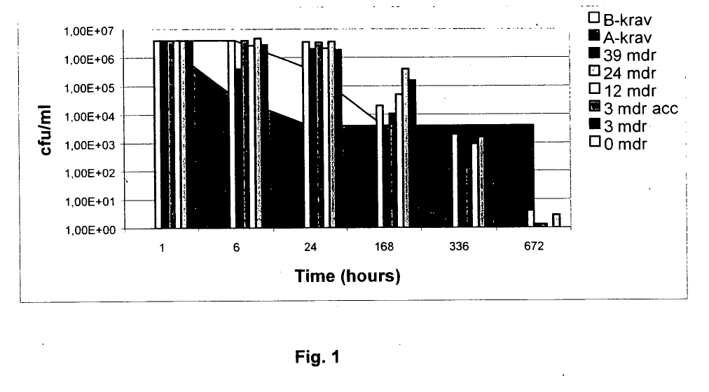

FIG. 1 shows the results of tolerance tests on pilot vaccine with the preservative TF-1 according to the present invention toward the germ S. aureus. It has surprisingly been found that a combination of at least two paraben esters selected from the group consisting of methyl, ethyl, propyl and butyl p-hydroxybenzoate act synergistically in combination with 2-phenoxyethanol and serves as an effective preservative (hereafter called TF-1) without affecting the immunogenic effect of the immunogen of the vaccine. In one aspect the present invention provides a vaccine formulation comprising an immunogen, a preservative and suitable pharmaceutically acceptable excipients for vaccines. The vaccine formulation according to the invention is characterized in that the preservative is a combination of at least two paraben esters and 2-phenoxyethanol. The paraben esters are selected from the group consisting of methyl, ethyl, propyl or butyl p-hydroxybenzoate.

The vaccine formulation according to the invention containing an immunogen can be prepared and marketed in the form of a suspension or a solution. The immunogen employed in the vaccine according to the invention may be effective against bacterial, viral, fungal, prion, or parasitic infections. A suitable immunogen is inactivated porcine parvovirus. Other suitable immunogens for vaccines are known to the person skilled in the art of formulating vaccine. In a preferred embodiment of the invention the vaccine formulation comprises inactivated porcine parvovirus.

In a preferred embodiment of the invention the preservative is a combination of methyl p-hydroxybenzoate, propyl p-hydroxybenzoate and 2-phenoxyethanol.

The preservative compounds are used in concentrations known to the person skilled in the art of formulating vaccine. In a preferred embodiment of the invention methyl p-hydroxybenzoate is used in a concentration of 0.01-2.5 mg/ml, ethyl p-hydroxybenzoate is used in a concentration of 0.01-0.7 mg/ml, propyl p-hydroxybenzoate is used in a concentration of 0.01-0.5 mg/ml or butyl p-hydroxybenzoate is used in a concentration of 0.01-0.16 mg/ml and 2-phenoxyethanol is used in a concentration of 0.4-26 mg/ml. In a more preferred embodiment of the invention methyl p-hydroxybenzoate is used in a concentration of 1.1-1.5 mg/ml, ethyl p-hydroxybenzoate is used in a concentration of 0.3-0.35 mg/ml, propyl p-hydroxybenzoate is used in a concentration of 0.17-0.3 mg/ml or butyl p-hydroxybenzoate is used in a concentration of 0.05-0.08 mg/ml and 2-phenoxyethanol is used in a concentration of 0.8-16 mg/ml. In yet a more preferred embodiment of the invention methyl p-hydroxybenzoate is used in a concentration of about 1.3 mg/ml, propyl p-hydroxybenzoate is used in a concentration of about 0.2 mg/ml and 2-phenoxyethanol is used in a concentration of about 1.0 mg/ml. If solubilizing agents known to a person skilled in the art of preparing vaccine formulations are used in the vaccine formulation, it may be possible to use higher concentrations of the preservatives.

The vaccine formulation additionally contains pharmaceutically acceptable excipients such as diluents, adjuvants, stabilisers, preservatives, buffers, surfactants, viscosity controlling agents, osmotic pressure controlling agents. Such excipients are all selected in accordance with conventional pharmaceutical practice in a manner understood by the persons skilled in the art of preparing vaccine formulations.

Diluents include water, aqueous buffer (such as buffered saline), alcohols and polyols (such as glycerol).

Adjuvants are normally used to enhance the immune-stimulating properties of an immunogen. In one embodiment of the invention at least one adjuvant is incorporated into the vaccine formulation according to the invention.

Suitable adjuvants are exemplified by but not limited to aluminium hydroxide gel, emulsigen, aluminium phosphate, calcium phosphate, oil/water emulsions, Quillaja saponin and ginsenoids. In a preferred embodiment of the invention the vaccine formulation comprises aluminium hydroxide gel and Quillaja saponin. Since the use of adjuvants in vaccines may be associated with drawbacks, e.g. unexpected systemic or local reactions, only a certain maximum amount should be injected per dose. Thus, the concentration of a certain adjuvant in a vaccine will be adjusted according to the volume of the dose of the particular vaccine to be injected. The person skilled in the art of preparing vaccine formulations will know how to adjust the concentration of the adjuvant in the vaccine formulation in order to adhere to the requirements set by the regulatory authorities. In a preferred embodiment of the invention aluminium hydroxide gel is used in a concentration of 0.5-5 mg aluminium/dose and Quillaja saponin is used in a concentration of 20 μg -1 mg/dose.

In another aspect the present invention provides a ready-to-use mixture of preservative and adjuvant. Since it is often a tedious part of the manufacturing process to prepare different stock solution, it may be very convenient to have access to a ready-to-use base stock solution comprising preservatives and adjuvant. In a preferred embodiment of the invention the base stock solution for the preparation of vaccines can be exemplified by but not limited to a mixture of aluminium hydroxide gel and/or Quillaja saponin and a combination of at least two paraben esters and 2-phenoxyethanol.

The invention is further explained and illustrated by the following examples which should not be contrued as limiting the scope of the invention in any respect.

### EXAMPLES

### Example 1

Preparation of a Preservative Stock Solution of TF-1 (2 Liters):

| | |
|---|---|
| Propyl-4-hydroxybenzoate | 40 g |
| Methyl-4-hydroxybenzoate | 60 g |
| 2-Phenoxy ethanol | 200 g (182 ml) |
| 96% Ethanol | up to 2 l |

The compounds are dissolved in an aliquot of 96% ethanol using magnetic stirring. Ethanol is added up to 2 liters. The solution is sterile filtered employing a Millipore Sterivex GV, 0.22 μm filter with a Durapore membrane. The solution is filled into sterile bottles and the bottles are closed with a cap.

Preparation of a Veterinary Vaccine (140 l)

| | |
|---|---|
| Inactivated porcine parvovirus [ca. 3 μg virion/ml] | 21898 ml |
| Aluminium hydroxide (2.0% Al(OH)₃) | 67760 ml |
| 2 M Glycine | 319 ml |
| Ultrafiltered water | 32258 ml |
| Phosphate-NaCl, pH 7, 2 | 13540 ml |
| 2% Quillaja saponin solution | 1752 ml |
| 2 M sodium thiosulphate | 680 ml |
| Preservative stock solution (TF 1) | 1400 ml |
| Antifoaming agent | 392 ml |

All the ingredients were mixed and stirred. The final vaccine formulation was filled into suitable containers and a stability study was conducted:

The vaccine was tested at 0, 1, 3, 6, 12, 24 and 39 months after formulation, and a test was also performed after storage at elevated temperature for 3 months.

### Results

Preservative challenge test

When testing for efficacy of antimicrobial preservation by challenging the vaccine preparation with a prescriped inoculum of four different test microorganisms, according to Ph.Eur. 3^{rd} Ed. It was seen, that the vaccine fulfilled the A-requirements in Ph.Eur 3^{rd} Ed with three of the microorganisms. The preservative was inadequate against S. aureus (6 h and 24 h) but at the end of the test period the requirements were fulfilled. This means, that the preservative is effective also against S. aureus, but it needs more time than the Ph.Eur allows (Table 1, FIG. 1)

TABLE 1
Tolerance tests on pilot vaccine with TF-1 stored during 39 months
at 4-6° C. Each circle indicate the test result according to the
respective period (6 h, 24 h, 7 days, 14 days and 28 days)

| Organism | Results |
|---|---|
| P. aeruginosa | The vaccine complies with the requirements of |
| | Ph. EU. 3^{rd} ED. |
| | A [symbol] [symbol] [symbol] [symbol] [symbol] |
| | B [symbol] [symbol] [symbol] [symbol] [symbol] |
| S. aureus | The vaccine does not comply with the |
| | requirements of Ph. EU. 3^{rd} ED. |
| | A ◯◯ [symbol] [symbol] [symbol] |
| | B [symbol] ◯◯ [symbol] [symbol] |
| C. albicans | The vaccine complies with the requirements of |
| | Ph. EU. 3^{rd} ED. |
| | A [symbol] [symbol] [symbol] [symbol] [symbol] |
| | B [symbol] [symbol] [symbol] [symbol] [symbol] |
| A. niger | The vaccine complies with the requirements of |
| | Ph. EU. 3^{rd} ED. |
| | A [symbol] [symbol] [symbol] [symbol] [symbol] |
| | B [symbol] [symbol] [symbol] [symbol] [symbol] |

◯: Ph. Eur. requirements not fulfilled; [symbol]
: No requirements; [symbol]
: Ph. Eur. requirements fulfilled.

According to Ph.Eur., 3rd Edition, these are the following criteria for accept of the efficacy of the preservative:

Log Reduction

| | | 6 h | 24 h | 7 days | 14 days | 28 days |
|---|---|---|---|---|---|---|
| Germ | A | 2 | 3 | — | — | NR* |
| | B | — | 1 | 3 | — | NI** |
| Fungi | A | — | — | 2 | — | NI |
| | B | — | — | — | 1 | NI |

*NR: no recover (meaning below limit of detection)
**NI: no increase (in relation to hitherto lowest amount).

Immunogenic Effects

It was found, that the immunogenic effect of the vaccine when vaccinating guinea pigs and pigs was not affected by the preservative. No unexpected systemic or local reactions were observed.

### Example 2

Composition of a Ready-to-use Base Stock Solution (Tf-1) Containing Aluminium Hydroxide Gel, Quillaia saponin and Preservatives

| | |
|---|---|
| Aluminium hydroxide (2.0% Al(OH)₃) | 6.3 mg/ml |
| Quillaja saponin | 0.25 mg/ml |
| Propyl-4-hydroxybenzoate | 0.20 mg/ml |
| Methyl-4-hydroxybenzoate | 1.3 mg/ml |
| 2-Phenoxyethanol | 1.0 mg/ml |

Composition of a Ready-to-use Base Stock Solution Containing Aluminium Hydroxide Gel A and Preservatives

| | |
|---|---|
| Aluminium hydroxide (2.0% Al(OH)₃) | 6.3 mg/ml |
| Propyl-4-hydroxybenzoate | 0.20 mg/ml |
| Methyl-4-hydroxybenzoate | 1.3 mg/ml |
| 2-Phenoxyethanol | 1.0 mg/ml |

Composition of a Ready-to-use Base Stock Solution Containing Saponin-Quil-A and Preservatives

| | |
|---|---|
| Quillaja saponin | 0.25 mg/ml |
| Propyl-4-hydroxybenzoate | 0.20 mg/ml |
| Methyl-4-hydroxybenzoate | 1.3 mg/ml |
| 2-Phenoxyethanol | 1.0 mg/ml |

I claim:

1. A vaccine formulation comprising an immunogen, a preservative and suitable pharmaceutically acceptable excipients for vaccines characterized in that the preservative is a combination of at least two paraben esters and 2-phenoxyethanol.

2. A vaccine formulation according to claim 1, wherein the paraben esters are selected from the group consisting of methyl, ethyl, propyl and butyl p-hydroxybenzoate.

3. A vaccine formulation according to claim 1, wherein the preservative is a combination of methyl p-hydroxybenzoate, propyl p-hydroxybenzoate and 2-phenoxy ethanol.

4. A vaccine formulation according to claim 1, wherein the excipients are selected from the group consisting of diluents, stabilizers, adjuvants, preservatives, buffers, surfactants, viscosity controlling agents and osmotic pressure controlling agents.

5. A vaccine formulation according to claim 1, which further comprises aluminium hydroxide gel as an adjuvant.

6. A vaccine formulation according to claim 1, which further comprises Quillaja saponin as an adjuvant.

7. A vaccine formulation according to claim 1, wherein methyl p-hydroxybenzoate if present, is used in a concentration of 0.01-2.5 mg/ml, ethyl p-hydroxybenzoate if present, is used in a concentration of 0.01-0.7 mg/ml, propyl p-hydroxybenzoate if present, is used in a concentration of 0.01-0.5 mg/ml or butyl p-hydroxybenzoate if present, is used in a concentration of 0.01-0.16 mg/ml and 2-phenoxyethanol is used in a concentration of 0.4-26 mg/ml.

8. A vaccine formulation according to claim 7, wherein methyl p-hydroxybenzoate is used in a concentration of about 1.3 mg/ml, propyl p-hydroxybenzoate is used in a concentration of about 0.2 mg/ml and 2-phenoxyethanol is used in a concentration of about 1.0 mg/ml.

9. A base stock solution of excipients for the preparation of vaccines comprising an aluminium hydroxide gel and a combination of at least two paraben esters and 2-phenoxyethanol.

10. A base stock solution of excipients for the preparation of vaccines comprising Quillaja saponin and a combination of at least two paraben esters and 2-phenoxyethanol.

11. A basic stock solution of excipients for the preparation of vaccines comprising an aluminium hydroxide gel, Quillaja saponin and a combination of at least two paraben esters and 2-phenoxyethanol.
